(19) United States
(12) Patent Application Publication
(10) Pub. No.: US 2014/0133626 A1
(43) Pub. Date: May 15, 2014

(54) X-RAY IMAGING APPARATUS AND X-RAY IMAGING METHOD

(71) Applicant: SAMSUNG ELECTRONICS CO., LTD., Suwon-si (KR)

(72) Inventors: Kwang Eun JANG, Busan (KR); Jong Ha LEE, Hwaseong-si (KR); Young Hun SUNG, Hwaseong-si (KR); Kang Eui LEE, Seoul (KR); Jae Hak LEE, Yongin-si (KR)

(73) Assignee: SAMSUNG ELECTRONICS CO., LTD., Suwon-si (KR)

(21) Appl. No.: 14/055,118

(22) Filed: Oct. 16, 2013

(30) Foreign Application Priority Data
Nov. 9, 2012 (KR) 10-2012-0126901

(51) Int. Cl. A61B 6/00

(52) U.S. Cl. CPC A61B 6/542
USPC 378/62

(57) ABSTRACT

The X-ray imaging method includes setting imaging conditions according to characteristics of an object and performing X-ray imaging of the object based on the set imaging conditions. The imaging conditions include at least one of an imaging angle, a number of imaging operations, and an imaging position.

### CROSS-REFERENCE TO RELATED APPLICATION

This application claims priority from Korean Patent Application No. 10-2012-0126901, filed on Nov. 9, 2012 in the Korean Intellectual Property Office, the disclosure of which is incorporated herein by reference in its entirety.

### BACKGROUND

1. Field

Apparatuses and methods consistent with exemplary embodiments relate to an X-ray imaging apparatus that irradiates an object with X-rays to generate an X-ray image and has an automatic exposure control (AEC) function and an X-ray imaging method.

2. Description of the Related Art

An X-ray imaging apparatus is an apparatus that diagnoses lesions inside an object by irradiating the object with X-rays and acquiring an image from X-rays having passed through the object, and X-ray imaging apparatuses to capture an image concentrated on any one area of a human body have been released.

One example of an X-ray imaging apparatus to capture an image concentrated on any one area of a human body is a mammography system. The mammography system serves to acquire a two-dimensional (2D) X-ray image of a breast by irradiating a compressed breast with X-rays.

The mammography system may be advantageous to detect lesions inside a breast at low cost, but has difficulty in distinguishing mass from other tissues in the acquired 2D X-ray image. In particular, upon imaging of a dense breast, there is a limit for an inspector to distinguish lesions from internal tissues of the breast only using the acquired 2D X-ray image because the tissues overlap one another.

To overcome such limit, a tomosynthesis system has been devised, which captures images of a compressed breast at different angles while moving an X-ray generator that generates X-rays, thereby acquiring a three-dimensional (3D) X-ray image.

The tomosynthesis system captures images of a breast at various angles and composes 2D images acquired through respective captured images to form a 3D image. Thus, although the tomosynthesis system easily detects lesions as compared to the mammography system, the tomosynthesis system may require a plurality of X-ray emissions, which causes a problem in regard to patient radiation exposure.

Accordingly, in the tomosynthesis system, it may be important to reduce patient radiation exposure while detecting lesions of the patient without errors by appropriately setting imaging conditions, such as an imaging angle of a breast.

### SUMMARY

Exemplary embodiments may address at least the above problems and/or disadvantages and other disadvantages not described above. Also, the exemplary embodiments are not required to overcome the disadvantages described above, and an exemplary embodiment may not overcome any of the problems described above.

One or more of exemplary embodiments provide an X-ray imaging apparatus and an X-ray imaging method in which imaging conditions are set by applying tissue characteristics of an object.

In accordance with an aspect of an exemplary embodiment, an X-ray imaging method includes setting imaging conditions according to characteristics of an object and performing X-ray imaging of the object based on the set imaging conditions, wherein the imaging conditions include at least one of an imaging angle, the number of imaging operations, and an imaging position.

The imaging angle may be set to a greater value as a compressed thickness of the object increases, and may be set to a smaller value as the compressed thickness of the object decreases.

The imaging angle may be set to a greater value as a density of the object increases, and may be set to a smaller value as the density of the object decreases.

The number of imaging operations may be set to a greater value as a compressed thickness of the object increases, and may be set to a smaller value as the compressed thickness of the object decreases.

The number of imaging operations may be set to a greater value as a density of the object increases, and may be set to a smaller value as the density of the object decreases.

The imaging position may include a plurality of imaging positions, a distance between two adjacent imaging positions among the imaging positions being differently set in a central region and a peripheral region of an imaging angle region.

The distance between two adjacent imaging positions may be narrower in the central region than in the peripheral region.

At least one of a magnitude of a tube voltage, a filter, and an anode material may be differently set according to the imaging position.

The at least one of a magnitude of a tube voltage, a filter, and an anode material may be differently set in the central region and the peripheral region.

The imaging conditions may further include at least one of a magnitude of a tube voltage, a filter material, and an anode material.

In accordance with another aspect of an exemplary embodiment, an X-ray imaging method includes setting imaging conditions according to characteristics of an object and performing X-ray imaging of the object based on the set imaging conditions, wherein the characteristics of the object include at least one of thickness and density of the object.

The density may be determined using a histogram of a pre-shot image for the object.

In accordance with another aspect of an exemplary embodiment, an X-ray imaging apparatus includes an X-ray generator to generate X-rays and irradiate an object with the generated X-rays, an X-ray detector to detect X-rays having passed through the object, an image processor to acquire an image of the object from the detected X-rays, and a controller to set imaging conditions according to characteristics of the object and control X-ray imaging of the object based on the set imaging conditions, wherein the imaging conditions include at least one of an imaging angle, the number of imaging operations, and an imaging position.

The imaging angle may be set to a greater value as a compressed thickness or a density of the object increases, and may be set to a smaller value as the compressed thickness or the density of the object decreases.

The number of imaging operations may be set to a greater value as a compressed thickness or a density of the object increases, and may be set to a smaller value as the compressed thickness or the density of the object decreases.

The imaging position may include a plurality of imaging positions, a distance between two adjacent imaging positions among the imaging positions being differently set in a central region and a peripheral region of an imaging angle region.

The distance between two adjacent imaging positions may be narrower in the central region than in the peripheral region.

At least one of a magnitude of a tube voltage, a filter, and an anode material may be differently set according to the imaging position.

The at least one of a magnitude of a tube voltage, a filter, and an anode material may be differently set in the central region and the peripheral region.

### BRIEF DESCRIPTION OF THE DRAWINGS

The above and/or other aspects will become more apparent by describing certain exemplary embodiments, with reference to the accompanying drawings, in which:

FIG. 1 is a perspective view of an X-ray imaging apparatus according to an exemplary embodiment;

FIG. 2 is a side view of the X-ray imaging apparatus of FIG. 1;

FIG. 3 is a view illustrating rotation of an arm of the X-ray imaging apparatus illustrated in FIGS. 1 and 2;

FIG. 4 is a control block diagram of the X-ray imaging apparatus according to an exemplary embodiment;

FIG. 5 is a histogram of a pre-shot image;

FIG. 6A is a table illustrating imaging angles depending on the thickness of a breast;

FIG. 6B is a table illustrating imaging angles depending on the density of a breast;

FIG. 6C is a table illustrating imaging angles depending on the thickness and density of a breast;

FIG. 7A is a table illustrating the number of imaging operations depending on the thickness of a breast;

FIG. 7B is a table illustrating the number of imaging operations depending on the density of a breast;

FIG. 7C is a table illustrating the number of imaging operations depending on the thickness and density of a breast;

FIG. 8 is a view illustrating imaging positions;

FIGS. 9A and 9B are tables illustrating tube voltage value/filter material depending on the thickness and density of a breast; and

FIG. 10 is a flowchart illustrating an X-ray imaging method according to an exemplary embodiment.

### DETAILED DESCRIPTION

Certain exemplary embodiments are described in greater detail below with reference to the accompanying drawings.

In the following description, the same drawing reference numerals are used for the same elements even in different drawings. The matters defined in the description, such as detailed construction and elements, are provided to assist in a comprehensive understanding of exemplary embodiments. Thus, it is apparent that exemplary embodiments can be carried out without those specifically defined matters. Also, well-known functions or constructions are not described in detail since they would obscure exemplary embodiments with unnecessary detail.

Hereinafter, exemplary embodiments of an X-ray imaging apparatus and an X-ray imaging method will be described in detail. In the following description, the X-ray imaging apparatus will be described as a tomosynthesis system.

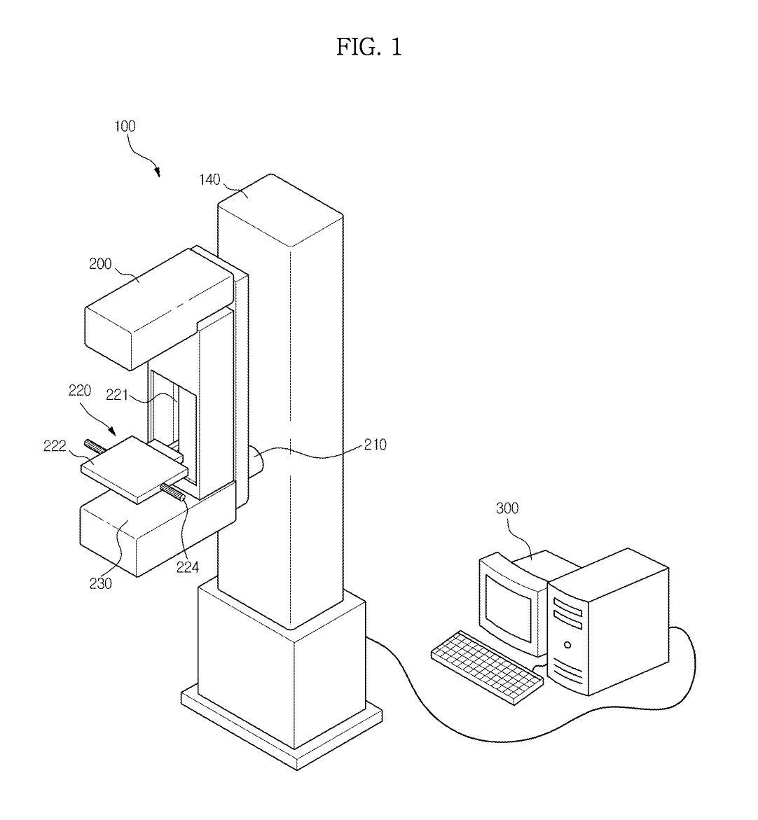

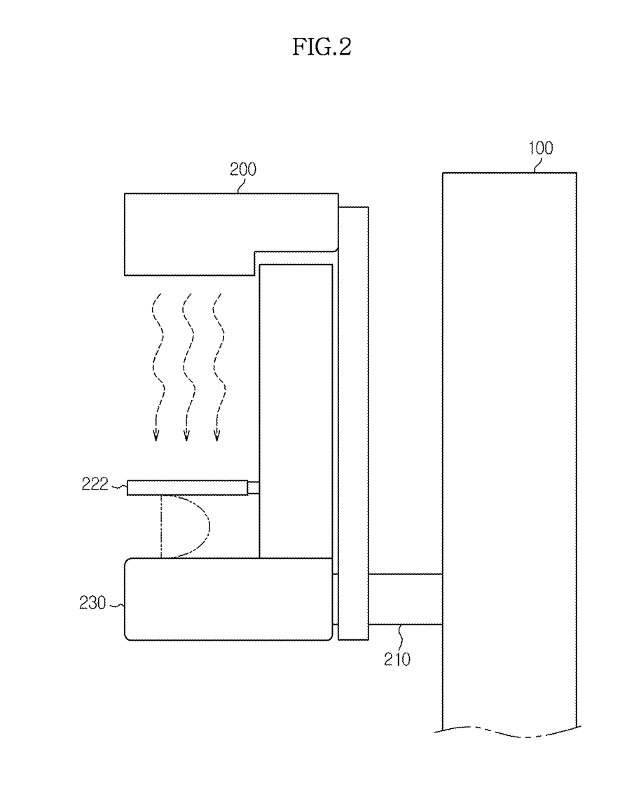

FIGS. 1 to 3 illustrate an X-ray imaging apparatus according to an exemplary embodiment.

Referring to FIGS. 1 to 3, the X-ray imaging apparatus includes a gantry 100 and an inspector workstation 300.

The gantry 100 includes a main body 140 and an arm 200 that is rotatable about a rotating shaft 210 fixed at the main body 140. A variety of electronic parts needed for X-ray generation and connection wires thereof are disposed in the main body 140.

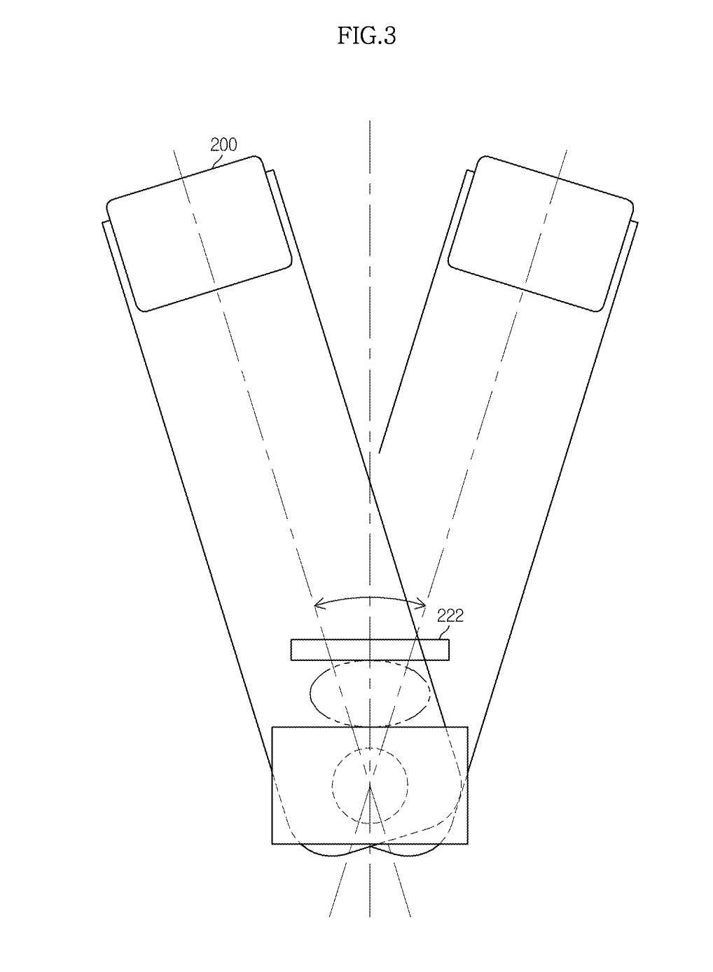

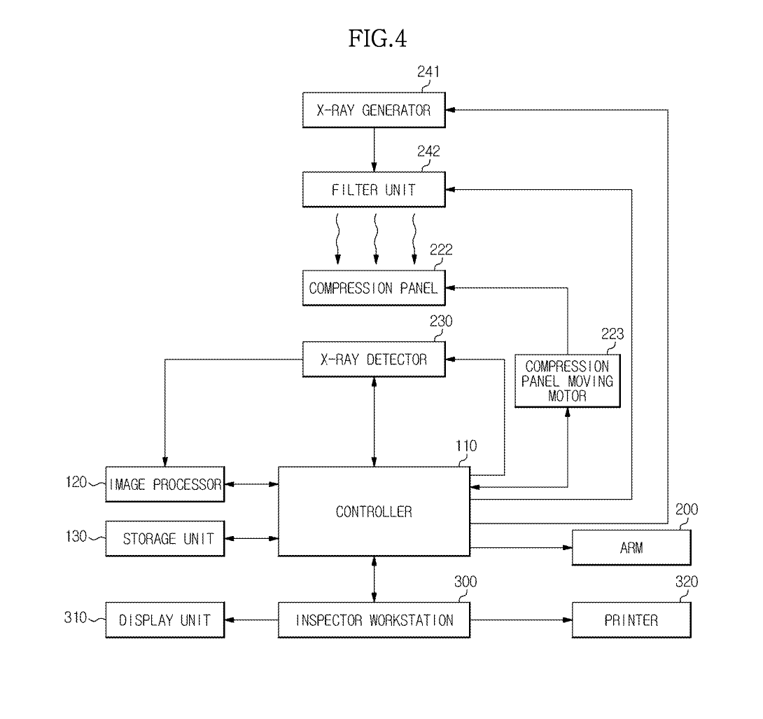

The arm 200 may include an X-ray generator 241 (see FIG. 4) to generate X-rays and irradiate an object, i.e., a breast with the generated X-rays and a filter unit 242 (FIG. 4). The X-ray generator 241 may rotate together with the arm 200 according to rotation of the arm 200. As illustrated in FIG. 3, the X-ray generator 241 may rotate leftward or rightward about a vertical center line (a straight line passing a center of the arm 200 and a center of an X-ray detector which will be described below). In this regard, an angle of leftward or rightward rotation of the X-ray generator 241 about the vertical center line may be determined as an imaging angle. The imaging angle will be described below in detail.

X-rays are generated as a tube voltage is applied to a cathode (not shown) of an X-ray tube (not shown) and tube current flows through a filament (not show) of the X-ray tube. In this case, the tube voltage and tube current may differ between X-ray imaging apparatuses. In the current exemplary embodiment, a tube voltage of 20 to 50 kVp (Vp: peak voltage) and a tube current of 10 to 120 mAs (milliampere sec) are given by way of example.

The tube voltage has an effect on the quantity of X-rays generated in the X-ray tube and energy (that determines transmittance). If the tube voltage increases, the peak of an X-ray spectrum increases and movement in a high-energy direction occurs. This means that the number of photons generated in the X-ray tube increases and an overall average energy of the photons also increases as a tube voltage increases.

The tube current has an effect on the quantity of X-rays generated in the X-ray tube. If the tube current increases, the peak of an X-ray spectrum increases and movement in a low-energy or high-energy direction does not occur. This means that the number of photons generated in the X-ray tube increases as tube current increases.

An anode (not shown) is a portion with which electrons emitted from the cathode (filament) collide to generate X-rays. The anode is configured to be replaced with an anode of another material when X-rays are generated, as desired. In this regard, an X-ray spectrum of X-rays differs according to a constituent material of the anode. For example, if the anode is formed of tungsten, an X-ray spectrum generated from the anode exhibits a gentle ascending and descending graph, the peak of which is at about 24 keV (electron volts) under the condition of a tube voltage of 42 kVp. If the anode is formed of molybdenum, an X-ray spectrum generated from the anode exhibits a spike-shaped graph, the peak of which is about 17 keV under the condition of a tube voltage of 42 kVp.

The filter unit 242 (see FIG. 4) includes at least one filter installed such that one filter may be manually or automatically replaced with another filter. When X-rays pass through the filter, the peak of an X-ray spectrum is lowered and movement in a high-energy direction occurs. Thus, the filter serves to reduce the number of photons (in particular, low-energy photons) and increase the average energy of photons. In this case, a reduction rate in the number of photons and an increase rate in the energy of photons differ according to the kind of the filter.

The arm 200 may be provided at a front side thereof with an X-ray detector 230 to detect X-rays having passed through an object, e.g., a breast and a compressor 220 to compress the object towards the X-ray detector 230. The X-ray detector 230 is fixed to the rotating shaft 210 of the arm 200. The compressor 220 includes a compression panel 222 and a compression panel guide unit 221.

The compression panel 222 is movable upward or downward and is moved downward to compress the object. The compression panel 222 may be manually moved by an operator using the compression panel handle 224 or automatically moved using a compression panel moving motor 223 (see FIG. 4) installed in the compression panel guide unit 221.

The compression panel guide unit 221 serves to guide the compression panel 222 during movement of the compression panel 222, and the compression panel moving motor 223 (see FIG. 4) to move the compression panel 222 is installed in the compression panel guide unit 221.

The inspector workstation 300 displays or stores an X-ray image transmitted from the gantry 100.

FIG. 4 is a control block diagram of the X-ray imaging apparatus according to an exemplary embodiment.

As illustrated in FIG. 4, the X-ray imaging apparatus, in addition to the constituent elements illustrated in FIGS. 1 to 3, includes an image processor 120 to acquire an X-ray image from X-rays detected by the X-ray detector 230, a storage unit 130 in which a plurality of imaging condition tables is stored, a controller 110 to control general operations of the X-ray imaging apparatus, a display unit 310 to display an X-ray image, and a printer 320 to print the X-ray image, the display unit 310 and the printer 320 being connected to the inspector workstation 300.

The image processor 120 reads out electric signals of the X-ray detector 230 to acquire an image signal. The image processor 120 generates an X-ray image via reversion of the image signal (for example, via flat field correction). The image processor 120 generates a histogram of the acquired X-ray image. The image histogram is a graph representing brightness distribution of the X-ray image.

In the current exemplary embodiment, a breast as the object is located in front of a muscle bed, and consists of fibrous tissues constituting the periphery of the breast for shape maintenance, adipose tissues distributed throughout the breast, mammary tissues for generation of breast milk, vascular tissues as movement passage of breast milk, etc. Of these tissues, tissues related to generation and supply of breast milk, such as mammary tissues and vascular tissues as movement passages of breast milk, are referred to as breast parenchymal tissues. Here, the parenchymal tissues are mainly composed of protein components.

Protein has a greater density than fat and absorbs a great quantity of X-rays than fat when exposed to X-rays. Thus, when a breast having a great quantity of parenchymal tissues is irradiated with X-rays to acquire an X-ray image and the X-ray image is represented by a histogram, a graph having a low left side and a high right side is drawn. Conversely, when a breast having a great quantity of fat tissues is irradiated with X-rays to acquire an X-ray image and the X-ray image is represented by a histogram, a graph having a high left side and a low right side is drawn.

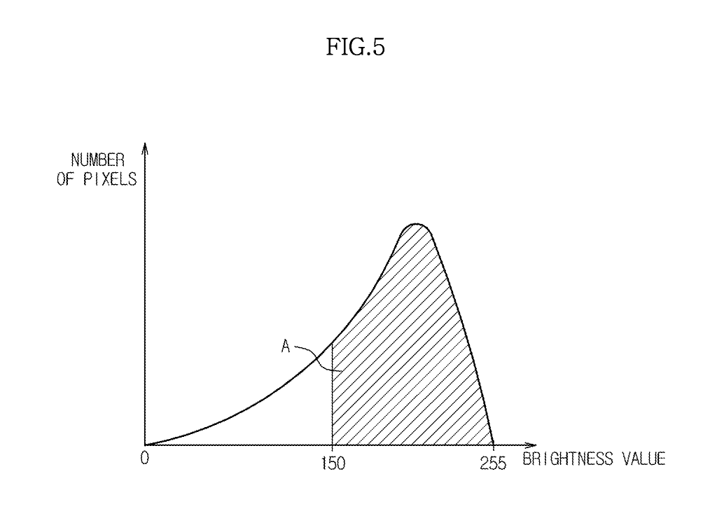

FIG. 5 is a histogram of an X-ray image. In FIG. 5, the X-axis represents a brightness value from 0 to 255 and the Y-axis represents the number of pixels corresponding to each brightness value. As mentioned above, as the brightness value approaches 0, this means that pixels corresponding to the brightness value become darker. Conversely, as the brightness value approaches 255, this means that pixels corresponding to the brightness value become brighter.

Referring to FIG. 5, it may be confirmed that the number of bright pixels is greater than the number of dark pixels. Accordingly, it may be confirmed that a breast represented by the image histogram of FIG. 5 contains more parenchymal tissues than fat tissues.

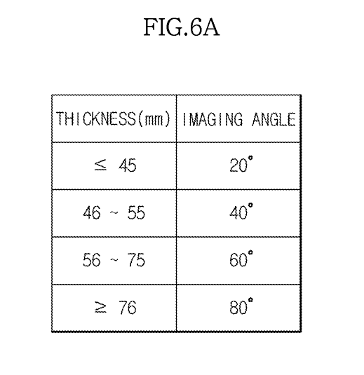

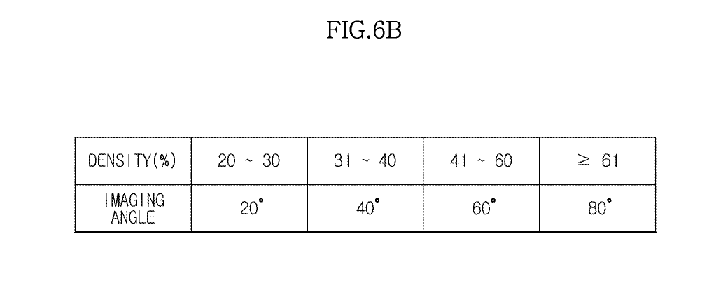

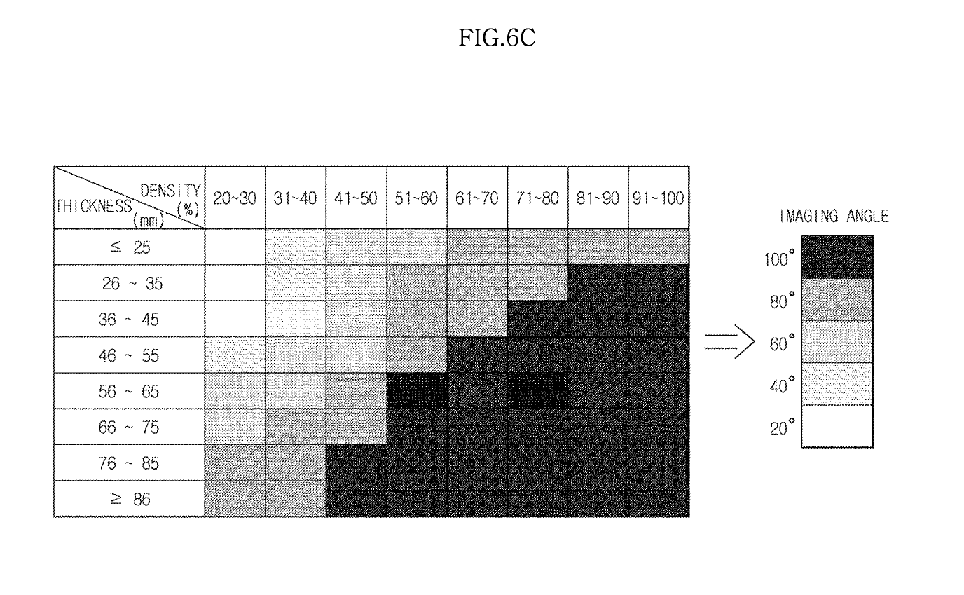

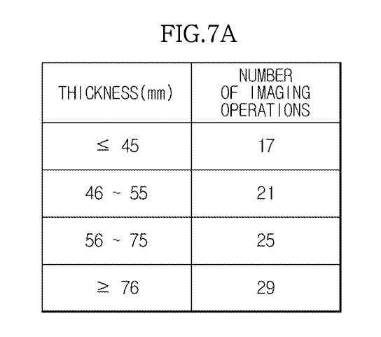

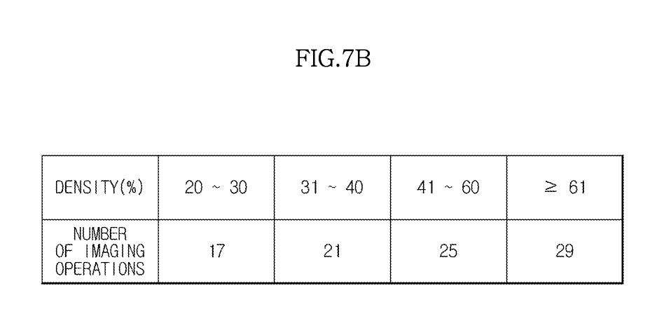

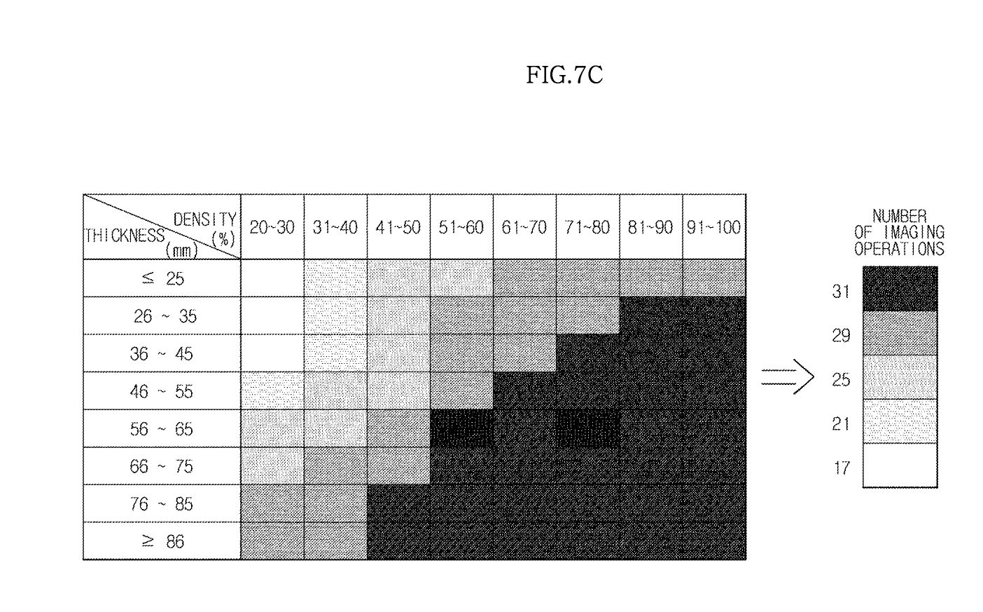

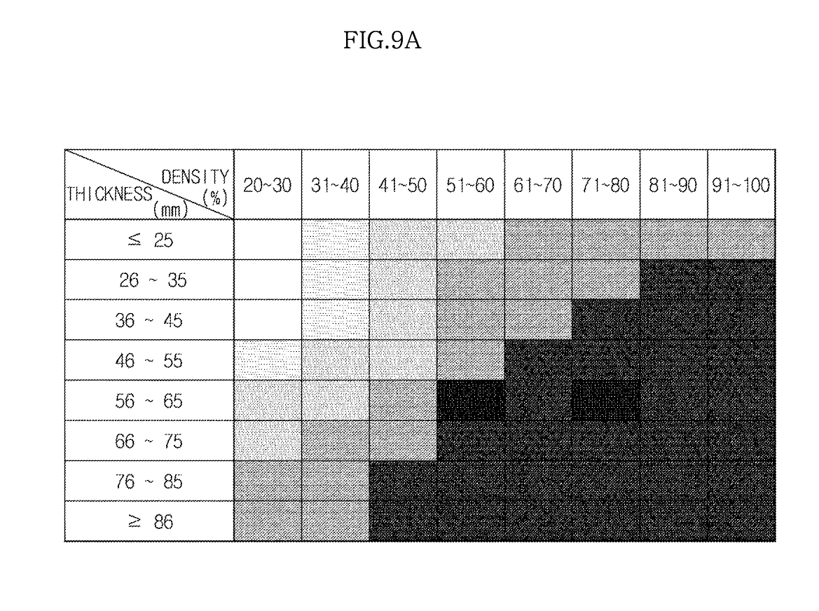

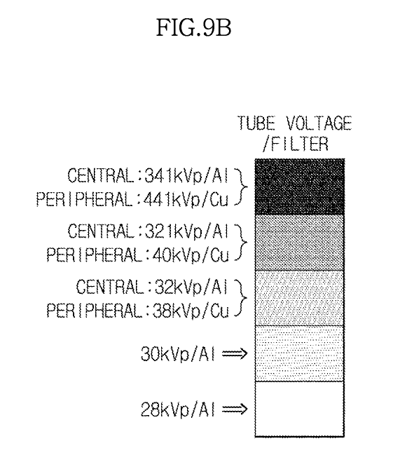

The storage unit 130 stores imaging condition tables upon X-ray imaging. Imaging conditions are differently set according to at least one of the thickness and density of a breast compressed by the compression panel 222. The imaging conditions include an imaging angle, the number of imaging operations, an imaging position, a tube voltage, a filter (kinds of a filter material), an anode (kinds of an anode material), etc. Thus, the storage unit 130 stores a table regarding imaging angles (see FIG. 6C), a table regarding the number of imaging operations (see FIG. 7C), a table regarding an imaging position, a tube voltage/filter table (see FIGS. 9A and 9B), and an anode table (not shown).

The thickness of a breast may be acquired by detecting a position of the compression panel 222. The position of the compression panel 222 may be detected by a sensor (not shown) attached to the compression panel guide unit 221 to detect the position of the compression panel 222, or by monitoring the movement of the compression panel moving motor 223 that moves the compression panel 222.

The density of a breast refers to the percentage of parenchymal tissues in the breast. The density of a breast may be acquired using various methods. In one example, the density of a breast may be acquired by calculating a ratio of the overall area of a breast to the area of parenchymal tissues displayed in an X-ray image.

Here, the overall area of the breast may be acquired via integration of an X-ray image histogram, and the area of parenchymal tissues may be acquired via integration of a particular region where a brightness value of a pixel is equal to or greater than a reference value (for example, a region A (FIG. 5) where the brightness value is 150 or more within a range of 0 to 255 in the above graph. In addition, the density of a breast may be calculated by (an integral value of a region where a brightness value is a reference value or more/an integral value of the overall image histogram)*100(%).

The imaging angle refers to a range of leftward and rightward rotation of the X-ray generator 241 to emit X-rays and, in the current exemplary embodiment, indicates (a maximum angle between a vertical center line and a straight line passing the center of a surface of the X-ray detector 230 and the X-ray generator 241)*2. For example, assuming that the imaging angle is 100°, the X-ray generator 241 irradiates an object, i.e., a breast with X-rays while rotating between positions in which the straight line passing the X-ray generator 241 and the center of the X-ray detector 230 has an angle of 50° with respect to the vertical center line leftward and rightward.

The imaging angle is changed according to the thickness of a breast as illustrated in an imaging angle table of FIG. 6A. That is, the imaging angle increases as a breast thickness increases, and decreases as a breast thickness decreases. For example, the imaging angle is 40° if the breast thickness is within a range of 46 to 55 mm, but is 60° if the breast thickness is within a range of 56 to 75 mm.

The imaging angle is changed according to the density of a breast as illustrated in an imaging angle table of FIG. 6B. That is, the imaging angle increase as a breast density increases, and decreases as the breast density decreases. For example, the imaging angle is 40° if the breast density is within a range of 31 to 40%, but is 60° if the breast density is within a range of 41 to 60%.

The imaging angle is changed according to both the thickness and density of a breast as illustrated in an imaging angle table of FIG. 6C. That is, the imaging angle increases as a breast thickness and density increase, and decreases as a breast thickness and density decrease. For example, the imaging angle is 40° if the breast thickness and density are within a range of 26 to 35 mm and within a range of 31 to 40%, respectively, but is 60° if the breast thickness and density are within a range of 36 to 45 mm and within a range of 41 to 50%, respectively.

By changing the imaging angle based on at least one of the thickness and density of a breast, imaging may be performed, at a wide angle, even on a thick and dense breast, which enables acquisition of 3D X-ray images having low blur and high resolution in a depth direction of a breast.

The number of imaging operations is the number of X-ray emission operations performed while the X-ray generator 241 rotates within the above-described imaging angle range. For example, if the number of imaging operations is 21, the X-ray generator 241 irradiates an object, i.e., a breast with X-rays twenty-one times while rotating within the imaging angle range. In this case, an X-ray image is acquired whenever X-rays are emitted, and consequently the number of imaging operations is equal to the number of X-ray images acquired by imaging. Accordingly, in the current exemplary embodiment, it is regarded that setting of the number of imaging operations is equal to setting of the number of X-ray images to be acquired.

The number of imaging operations is changed according to the thickness of a breast as illustrated in a table regarding the number of imaging operations of FIG. 7A. That is, the number of imaging operations increases as a breast thickness increases, and decreases as the breast thickness decreases. For example, the number of imaging operations is 21 if the breast thickness is within a range of 46 to 55 mm, but is 25 if the breast thickness is within a range of 56 to 75 mm.

The number of imaging operations is changed according to the density of a breast as illustrated in a table regarding the number of imaging operations of FIG. 7B. That is, the number of imaging operations increases as a breast density increases, and decreases as the breast density decreases. For example, the number of imaging operations is 21 if the breast density is within a range of 31 to 40%, but is 25 if the breast density is within a range of 41 to 60%.

The number of imaging operations is changed according to both the thickness and density of a breast as illustrated in a table regarding the number of imaging operations of FIG. 7C. That is, the number of imaging operations increases as a breast thickness and density increase, and decreases as the breast thickness and density decrease. For example, the number of imaging operations is 21 if the breast thickness and density are within a range of 26 to 35 mm and within a range of 31 to 40%, respectively, but is 25 if the breast thickness and density are within a range of 36 to 45 mm and within a range of 41 to 50%, respectively.

By changing the number of imaging operations based on at least one of the thickness and density of a breast, a greater number of X-ray images are acquired even in the case of a thick and dense breast, which enables acquisition of 3D X-ray images having a high resolution.

An imaging position refers to a position where the X-ray generator 241 emits X-rays. Basically, a distance between two imaging positions is set to a value acquired by dividing the imaging angle by the number of imaging operations. For example, if the imaging angle is 50° and the number of imaging operations is 25, the distance between two imaging positions is 50/25=2°. Therefore, positions that are sequentially arranged from an imaging position on the vertical center line and equally spaced apart by 2° (an angle between a line passing one imaging position and the center of a surface of the X-ray detector 230 and a line passing another imaging position and the center of a surface of the X-ray detector 230) are imaging positions.

Alternatively, distances between every two imaging positions may differ. A distance between two imaging positions in a central region of the imaging angle (i.e., a region within which an angle between the vertical center line and a straight line passing the center of a surface of the X-ray detector 230 and the X-ray generator 241 is a reference angle leftward and rightward, e.g., in the following embodiment, a region within a range of 12.5° leftward and rightward) may differ from a distance between two imaging positions in a peripheral region of the imaging angle (a region except for the central region of an imaging angle region.

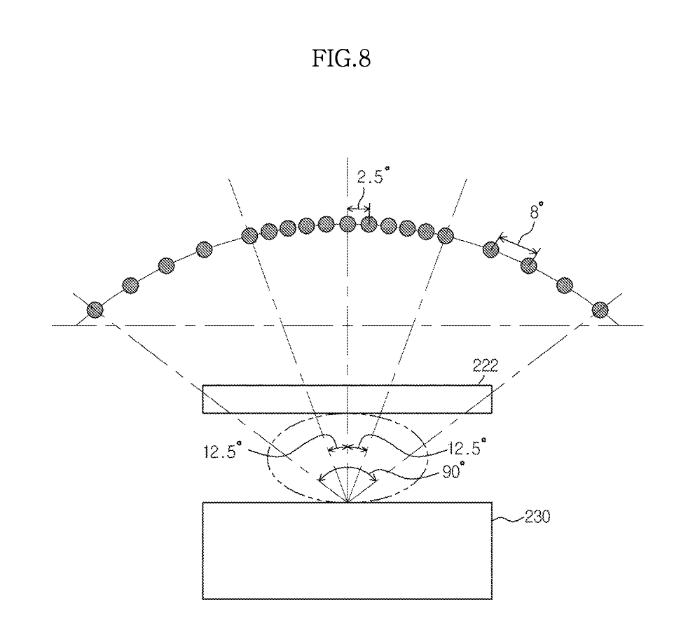

For example, as illustrated in FIG. 8, if the imaging angle is 90° and the number of imaging operations is 19, X-rays are emitted eleven times at imaging positions (from No. 5 to No. 15) that are sequentially arranged from the imaging position on the vertical center line and equally spaced apart by 2.5° to acquire eleven X-ray images within a range of 12.5° leftward and rightward (central region) on the basis of a vertical center line. Also, X-rays are emitted eight times at imaging positions (from No. 1 to No. 4 and No. 16 to No. 19) that are sequentially arranged from outermost imaging positions on left and right sides of the central region and equally spaced apart by 8° to acquire eight X-ray images within a left range of 12.6° to 50° and a right range of 12.6° to 50° (peripheral region). In FIG. 8, each point represents an imaging position, and each number is an imaging position number.

The imaging positions may be determined based on various factors, for example, based on the thickness or density of a breast. For example, as the thickness or density of a breast increases, a distance between the imaging positions in the central region becomes narrower than a distance between the imaging positions in the peripheral region, which enables acquisition of a high-resolution X-ray image. The imaging position table may be prepared based on various factors, such as the density of a breast, etc.

The definitions and effects of the tube voltage and the filter material have been mentioned above in FIGS. 1 to 3. The tube voltage and the filter material have an effect on the energy of X-rays and the quantity of photons. Thus, the tube voltage and the filter material are changed if at least one of the thickness and density of a breast is changed, which enables acquisition of a high-resolution 3D X-ray image.

For example, the tube voltage/filter table of FIGS. 9A and 9B show that the tube voltage and the filter material are differently set upon imaging if the thickness and density of a breast are changed. That is, as illustrated in FIGS. 9 A and 9B, if the thickness of a breast is within a range of 26 to 35 mm and the density of a breast is within a range of 31 to 40%, the tube voltage is 30 kVp and the filter is formed of aluminum (Al). On the other hand, if the thickness of a breast is within a range of 36 to 45 mm and the density of a breast is within a range of 41 to 50%, the tube voltage is 30 kVp in the central region and is 38 kVp in the peripheral region and the filter is formed of Al in the central region and copper (Cu) in the peripheral region.

As illustrated in the tube voltage/filter table of FIGS. 9A and 9B, if a breast is thick and dense, the tube voltage and the filter material in the central region differ from the tube voltage and the filter material in the peripheral region during imaging. That is, if a breast is thick and dense, the tube voltage/filter table is constructed such that the central region and the peripheral region have different tube voltages and different filter materials.

By varying the tube voltage and the filter material based on at least one of the thickness and density of a breast, even if a breast is thick and dense, X-rays having energy and quantity of photons suitable for breast tissues are generated, which enables acquisition of a high-resolution X-ray image.

The definition and role of the anode has been mentioned above in FIGS. 1 to 3. If a material of the anode is changed as described above, the energy of X-rays is changed, and thus the material of the anode may be differently set based on the thickness and density of a breast. Accordingly, the anode table may also be prepared based on at least one of the thickness and density of a breast. For example, when a breast has a large thickness and a high density, anodes of different materials may be respectively used in the central region and the peripheral region, thereby acquiring an X-ray image.

Although the above description exemplifies the tables stored in the storage unit 130, the preparation criteria of each table may differ from the above description. For example, although the current exemplary embodiment describes that, for example, the imaging angle is changed based on at least one of the thickness and density of a breast, each table may be prepared based on imaging conditions and other factors aside from the thickness and density of a breast. Numerical values listed in each table may be set differently from the current exemplary embodiment.

The controller 110 performs automatic exposure control (AEC). AEC is a function of automatically controlling X-ray exposure. More specifically, AEC is a function of controlling X-ray exposure by varying imaging conditions based on properties of breast tissues.

The aforementioned properties of tissues include the thickness and density of a breast. The controller 110 checks the thickness of a breast by receiving a sensed value from a sensor that senses a position of the compression panel 222, or by monitoring operation of the compression panel moving motor 223.

In addition, the controller 110 calculates the density of a breast by analyzing a pre-shot image that will be described hereinafter. The density of a breast, as described above, refers to the percentage of parenchymal tissues in a breast. The density of a breast may be calculated using various methods. For example, the density of a breast may be calculated by calculating a ratio of the overall area of a breast to the area of parenchymal tissues displayed in an X-ray image.

Here, the overall area of the breast may be acquired via integration of an X-ray image histogram, and the area of parenchymal tissues may be acquired via integration of a particular region where a brightness value of a pixel is equal to or greater than a reference value. In addition, the density of a breast may be calculated by (an integral value of a region where a brightness value is equal to or greater than a reference value/an integral value of the overall image histogram)*100(%).

If properties of tissues are checked, the controller 110 sets imaging conditions of main-shot that will be described hereinafter by referring to the respective tables of the storage unit 130. The main-shot refers to an imaging operation to generate an X-ray image for inspection of a breast, such as detection of lesions of a breast. The imaging conditions include the imaging angle, the number of imaging operations (i.e. the number of 2D X-ray images to be captured), the imaging position, the tube voltage, the filter material, and the anode material.

When the imaging conditions are set by performing AEC, the controller 110 performs main-shot.

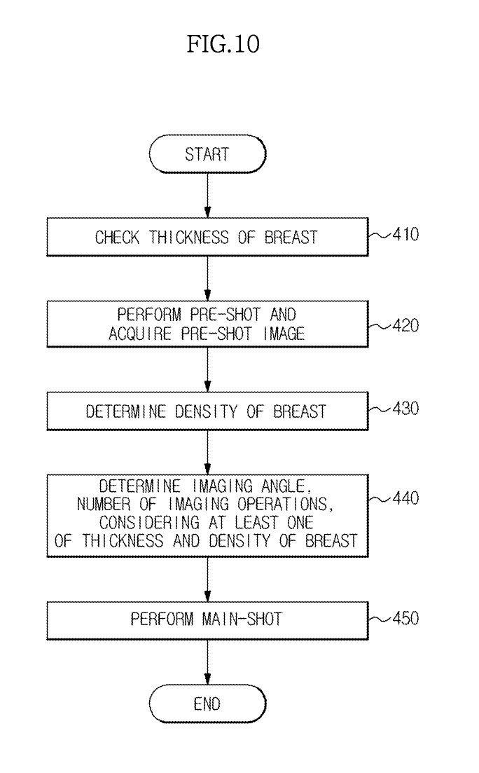

Hereinafter, an X-ray imaging method according to an exemplary embodiment will be described in detail with reference to FIG. 10.

If a breast of a patient is compressed through downward movement of the compression panel 222 in a state in which the breast is placed on the X-ray detector 230, the controller 110 checks the thickness of the breast (operation 410). The thickness of the breast, as described above, is acquired by a sensed value of the sensor that senses a position of the compression panel 222, or by monitoring rotation of the compression panel moving motor 223.

Next, the X-ray generator 241 emits X-rays to perform pre-shot, and the image processor 120 generates a 2D pre-shot image (operation 420). Pre-shot refers to an operation of capturing an X-ray image needed to acquire the density of a breast that is used as a criterion for setting of imaging conditions of main-shot.

Upon the pre-shot, the X-ray generator 241 emits X-rays once on the vertical center line (i.e., without leftward or rightward rotation), and the controller 110 sets imaging conditions, such as a tube voltage, tube current, etc., to emit low-dose X-rays to the breast.

Next, the image processor 120 generates a histogram of a pre-shot image. Once the image histogram is generated, the controller 110 acquires an integral value of the overall image histogram and an integral value of a region where the brightness value is equal to or greater than a reference value. A ratio between the two integral values, i.e. (the integral value of the region where the brightness value is equal to or greater than a reference value/the integral value of the overall image histogram)*100(%) is acquired and is determined as the density of a breast (operation 430).

Next, the controller 110 determines imaging conditions for main-shot based on at least one of the thickness and density of a breast (operation 440). The imaging conditions include an imaging angle, the number of imaging operations, an imaging position, a tube voltage, a filter material, and an anode material.

The controller 110 sets the imaging angle, the number of imaging operations, the imaging position, the tube voltage, the filter material, and the anode material with reference to tables regarding the imaging angle, the number of imaging operations, the imaging position, the tube voltage/filter, and the anode material stored in the storage unit 130.

Once the imaging conditions for main-shot are set, the X-ray generator 241 emits X-rays at the set imaging angle and times of the number of imaging operations set at the set imaging position, thereby performing main-shot. The image processor 120 generates a plurality of 2D main-shot images, and composes the main-shot images to generate a 3D X-ray image.

Although the main-shot is performed based on the set imaging conditions in the current exemplary embodiment, the set imaging conditions may be displayed to an inspector to provide the inspector with a chance to correct the displayed imaging conditions.

For example, the controller 110 may set the imaging conditions for the main-shot, and control the inspector workstation 300 to display the imaging conditions. In addition, under control of the controller 110, a message to inquire whether to correct the set imaging conditions may be displayed.

In addition, when the inspector inputs the changed imaging conditions to the inspector workstation 300, the controller 110 may receive the changed imaging conditions from the inspector workstation 300 and may control the main-shot based on the imaging conditions.

As is apparent from the above description, an X-ray image may be acquired by setting imaging conditions suitable for characteristics of an object.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An X-ray imaging method comprising:
setting imaging conditions according to characteristics of an object; and
performing X-ray imaging of the object based on the set imaging conditions, wherein the imaging conditions comprise at least one of an imaging angle, a number of imaging operations, and an imaging position.

2. The X-ray imaging method according to claim 1, wherein the imaging angle is set to a greater value as a compressed thickness of the object increases, and is set to a smaller value as the compressed thickness of the object decreases.

3. The X-ray imaging method according to claim 1, wherein the imaging angle is set to a greater value as a density of the object increases, and is set to a smaller value as the density of the object decreases.

4. The X-ray imaging method according to claim 1, wherein the number of imaging operations is set to a greater value as a compressed thickness of the object increases, and is set to a smaller value as the compressed thickness of the object decreases.

5. The X-ray imaging method according to claim 1, wherein the number of imaging operations is set to a greater value as a density of the object increases, and is set to a smaller value as the density of the object decreases.

6. The X-ray imaging method according to claim 1, wherein the imaging conditions comprise a plurality of imaging positions, and the method further comprises:
setting a first distance between two adjacent imaging positions, of the plurality of the imaging positions, disposed in a central region of an imaging angle region to a first value; and
setting a second distance between two adjacent imaging positions, of the plurality of the imaging positions, disposed in a peripheral region of the imaging angle region to the second value different from the first value.

7. The X-ray imaging method according to claim 6, wherein the first distance is narrower in the central region than the second distance in the peripheral region.

8. The X-ray imaging method according to claim 6, wherein at least one of a magnitude of a tube voltage, a filter, and an anode material is differently set according to the imaging position.

9. The X-ray imaging method according to claim 8, wherein the at least one of the magnitude of the tube voltage, the filter, and the anode material is differently set in the central region than that in and the peripheral region.

10. The X-ray imaging method according to claim 1, wherein the imaging conditions further comprise at least one of a magnitude of a tube voltage, a filter material, and an anode material.

11. An X-ray imaging method comprising:
setting imaging conditions according to characteristics of an object; and
performing X-ray imaging of the object based on the set imaging conditions, wherein the characteristics of the object comprise at least one of a thickness and a density of the object.

12. The X-ray imaging method according to claim 11, wherein the density is determined using a histogram of a pre-shot image of the object.

13. An X-ray imaging apparatus comprising:
an X-ray generator configured to generate and irradiate X-rays onto an object;
an X-ray detector configured to detect X-rays having passed through the object;
an image processor configured to acquire an image of the object from the detected X-rays; and
a controller configured to set imaging conditions according to characteristics of the object and control X-ray imaging of the object based on the set imaging conditions, wherein the imaging conditions comprise at least one of an imaging angle, a number of imaging operations, and an imaging position.

14. The X-ray imaging apparatus according to claim 13, wherein the imaging angle is set to a greater value as a compressed thickness or a density of the object increases, and is set to a smaller value as the compressed thickness or the density of the object decreases.

15. The X-ray imaging apparatus according to claim 13, wherein the number of imaging operations is set to a greater value as a compressed thickness or a density of the object increases, and is set to a smaller value as the compressed thickness or the density of the object decreases.

16. The X-ray imaging apparatus according to claim 13, wherein the imaging conditions comprise a plurality of imaging positions, and
a first distance between two adjacent imaging positions of the plurality of imaging positions disposed in a central region of an imaging angle region is set to a first value and second distance between two adjacent imaging positions of the plurality of imaging positions disposed in a peripheral region of the imaging angle region is set to a second value different from the first value.

17. The X-ray imaging apparatus according to claim 16, wherein the first distance is narrower in the central region than the second distance in the peripheral region.

18. The X-ray imaging apparatus according to claim 16, wherein at least one of a magnitude of a tube voltage, a filter, and an anode material is differently set according to the imaging position.

19. The X-ray imaging apparatus according to claim 18, wherein the at least one of the magnitude of the tube voltage, the filter, and the anode material is differently set in the central region than that in the peripheral region.
